(19) United States
(12) Patent Application Publication
(10) Pub. No.: US 2025/0191708 A1
(43) Pub. Date: Jun. 12, 2025

(54) SYSTEM AND METHOD OF INTEGRATION OF IN-PERSON AND IN-CLINIC SERVICE ENGINE PLATFORM WITH AN EMR SYSTEM

(71) Applicant: Teledact Inc., Thornhill (CA)

(72) Inventor: Tommy Tsz Him CHEUNG, Thornhill (CA)

(21) Appl. No.: 18/971,064

(22) Filed: Dec. 6, 2024

Related U.S. Application Data
(60) Provisional application No. 63/606,713, filed on Dec. 6, 2023.

(51) Int. Cl. G16H 10/60; G06Q 40/08; G16H 80/00

(52) U.S. Cl. CPC G16H 10/60; G06Q 40/08; G16H 80/00

(57) ABSTRACT

A system and method for integrating an in-person and virtual care Service Engine platform with an EMR system. The Service Engine platform provides connectivity and programmability across one or more EMR systems and provides communication across multiple communication channels including SMS, email, phone, text-to-voice and voice-to-text service. The Service Engine platform allows for full customization of its algorithms and analytics, integration with EMR and patient care workflow, driving better patient health outcomes and fulfilling physician care needs.

### CROSS REFERENCE TO RELATED APPLICATIONS

The application claims priority to and the benefit of U.S. Provisional Application Ser. No. 63/606,713, entitled “SYSTEM AND METHOD OF INTEGRATION OF IN-PERSON AND IN-CLINIC SERVICE ENGINE PLATFORM WITH AN EMR SYSTEM”, filed on Dec. 6, 2023 and is a US Continuation-in-part Utility Application of Ser. No. 18/192,598, entitled “SYSTEM AND METHOD OF INTEGRATION OF SERVICE ENGINE PLATFORM WITH AN EMR SYSTEM”, filed on Mar. 29, 2023, both of which are incorporated herein by reference in their entirety.

### BACKGROUND

The embodiments described herein relate to healthcare access navigation technologies, in particular, related to integrations with EMR systems.

Electronic medical records (EMR) are used by doctors and clinician offices to store patient medical information including individual contact information, and patient medical treatment and history. Currently, some EMR systems, such as Oscar Pro EMR, do not have a built-in communication system, such as an instant messaging or text messaging system to communicate with patients. Further, these systems also lack the ability to securely store messages in each patient's medical chart.

Patient care coordination and service navigation is complex and often involves a multitude of providers and organizations. There is a desire to provide a centralized application to assist patients and providers in care delivery to improve patient experience and also provide better health outcomes, in particular to care related to in-person and in-clinic consultations.

### SUMMARY

A system and method for integrating an in-person and in-service Service Engine platform with an EMR system. The Service Engine platform provides connectivity and programmability across one or more EMR systems and provides communication across multiple communication channels including SMS, email, phone, text-to-voice and voice-to-text service. The Service Engine platform allows for full customization of its algorithms and analytics, integration with EMR and patient care workflow, driving better patient health outcomes and fulfilling physician care needs.

### BRIEF DESCRIPTION OF THE DRAWINGS

FIG. 1 is a diagram illustrating the Service Engine.

FIG. 2 is a systems diagram illustrating components of the Service Engine system.

FIG. 3 is a systems diagram illustrating components of the In-Person Service Engine.

FIG. 4 is a process diagram illustrating a workflow of public vs insurance service coverage using a Service Engine.

FIG. 5 is a process diagram illustrating a workflow of a health service referral selection.

FIG. 6 is a process diagram illustrating a workflow of an In-Person In-clinic Service Engine.

### DETAILED DESCRIPTION

Practical software application is essential for patients seeking both virtual and in-person physician care and all services available from the associated health care providers. In particular, integration with existing electronic medical records (EMR) system can improve in-patient care and workflow. More information on EMR integration can be found in U.S. Provisional Patent Application Ser. No. 63/232,424, entitled “SYSTEM AND METHOD OF INTEGRATING TEXT MESSAGING READ NOTIFICATION WITH AN EMR SYSTEM”, filed on Aug. 12, 2021, US Provisional Patent Application Ser. No. 63/23247, entitled “SYSTEM AND METHOD OF INTEGRATING TEXT MESSAGING NOTIFICATION FEATURES WITH AN EMR SYSTEM”, filed on Aug. 12, 2021, and U.S. patent application Ser. No. 18/192,598, entitled “SYSTEM AND METHOD OF INTEGRATION OF SERVICE ENGINE PLATFORM WITH AN EMR SYSTEM”, filed on Mar. 29, 2023, the disclosures of which are incorporated herein by reference in their entirety.

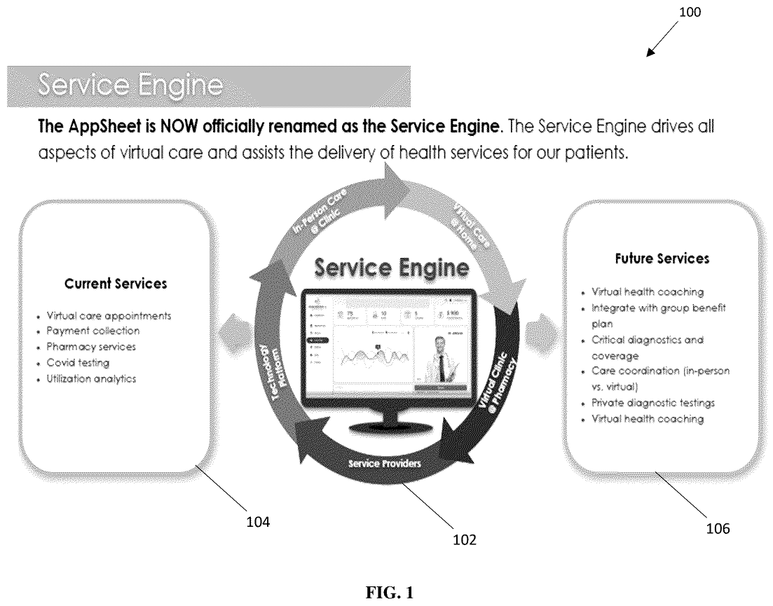

FIG. 1 is a diagram illustrating the Service Engine. According to FIG. 1, service engine system 100 is shown to be fully integrated with EMR and the patient care process, based on predefined algorithms and data analytics. Service Engine 102 is connected to current services 104 and future services 106. Patients and providers will receive prompts, notifications and guidance via various channels and systems that they interact with—EMR for physicians, mobile devices and SMS for patients, and other communication methods.

Service Engine 102 is now connected with over 250 pharmacies, clinics, health facilities, employer groups and other service organizations across the country, servicing thousands of patients each month. Algorithms have been set up to ensure service level targets and trigger necessary actions for physicians and other healthcare providers while they are servicing the patients.

According to FIG. 1, Service Engine 102 drives all aspects of virtual care and assists in the delivery of health services for patients. Service Engine 102 is a technology platform and connects to service providers. Furthermore, Service Engine 102 provides services for in-person care at the clinics, virtual care at home and virtual clinic support at pharmacies.

According to FIG. 1, current services 104 provided by Service Engine 102 include:
Physician virtual online and phone appointment and in-person requests and tracking
Prescription fulfillment and pharmacy service delivery
Covid testing data integration and result processing
Specialist care referrals
Uninsured and private service online payment collection
Laboratory and diagnostic testing request and documentation
Payment collection
Pharmacy services
COVID testing
Utilization analytics

In an embodiment, the Service Engine may be configured to support the following services 106:
Group Benefit Plans integration with major insurance service providers
Virtual health coaching with collaboration partners
EAP and chronic disease management coordination
Critical diagnostics and coverage
Care coordination (in person vs virtual)
Private diagnostic testing
Virtual health coaching

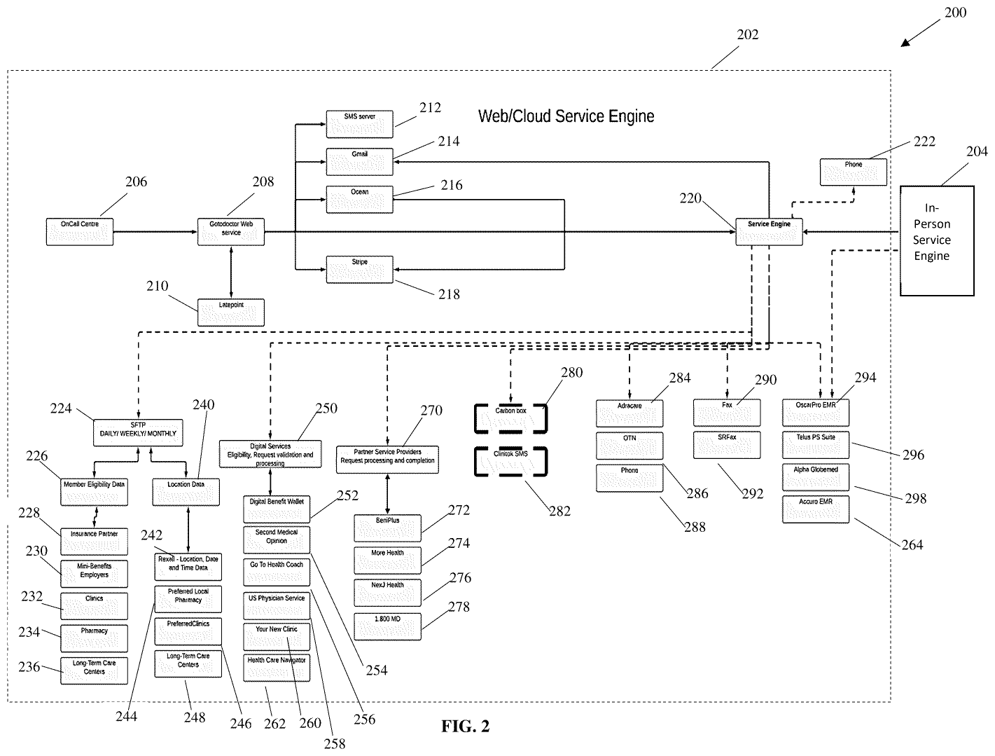

FIG. 2 is a systems diagram illustrating components of the Service Engine system. According to FIG. 2, Service Engine system 200 consists of web-based or cloud-based service engine 202 connected to an in-person service engine 204. Service engine 202 consists of multiple components including an initial input of virtual receptionists (e.g., OnCall Centre) 206. The information is sent to an online appointment systems (e.g., Gotodoctor Web Service) 208 with an asynchronous connection to appointment scheduling applications (e.g., Latepoint) 210.

According to FIG. 2, the system splits into multiple connections including an SMS server 212, an email server (e.g., Gmail) 214, connection to a health care platform (e.g., Ocean) 216, connection to payment service module (e.g., Stripe) 218 and connection to a Service Engine module 220. Service Engine module 220 will also provide connectivity to email server (e.g., Gmail) 214, a mobile phone (i.e., Android or Apple iOS) 222 and a plurality of complimentary services and applications.

According to FIG. 2, one of the complementary services and applications is a SFTP server 224. SFTP or Secure File Transfer Protocol is a secure file transfer protocol that uses secure shell encryption to provide a high level of security for sending and receiving file transfers. SFTP server 224 enables different duration connections, including daily, weekly or monthly, and enables data sharing of member eligibility data 226 and location data 240.

According to FIG. 2, member eligibility data 226 is connected to one or more insurance partners 226, mini-benefits employers 228, clinics 230, pharmacy 232 and long-term care centers 234.

According to FIG. 2, location data 240 is connected to one or more locations including Rexall pharmacy 242 which provides location, date and time data. Location data also includes connection to preferred local pharmacy 244, preferred clinics 246 and long-term care centers 248.

According to FIG. 2, further complementary services and applications include digital services 250. Digital services 250 checks for eligibility request validation and processing and is connected to one or more digital benefit wallets 252, a second medical opinion 254, a Go to Health coach 256, a US physician service 258, your new clinic 260 and a Health Care navigator service 262.

According to FIG. 2, further complementary services and applications include connection to partner service providers 270 which request processing and connection. Partner service providers 270 include connection to BeniPlus 272, More Health 274, NexJ Health 276 and 1-800 MD 278.

According to FIG. 2, complimentary services and application include connections to Carbon Box 280 and Clinitok SMS 282, Adracare 284, OTN 286, Phone 288, Fax 290, SRFax 292, OscarPro EMR 294, Telus PS Suite 296, Alpha Globemed 298 and Accuro EMR 264.

According to further disclosures, the Service Engine platform provides connectivity and programmability across one or more EMR systems and provides communication across multiple communication channels including short messaging service (SMS), email, phone, text-to-voice and voice-to-text services.

According to further disclosures, artificial intelligence (AI) and/or machine learning algorithms may be utilized to assist patients to identify the best healthcare access options (virtual vs. in-person visits, wait-time navigation to expedite care, recommendations to best course of action based on pre-screening and signs and symptoms presented). It will also provide better analytics and dashboard information for presentation to the end user to assist patients in their care journey. Currently, the care process is handled manually with clinic staff to triage, identify appropriate visit types and other recommendations. The Service Engine is the first of its kind that can navigate among all available public and private health services in Canada.

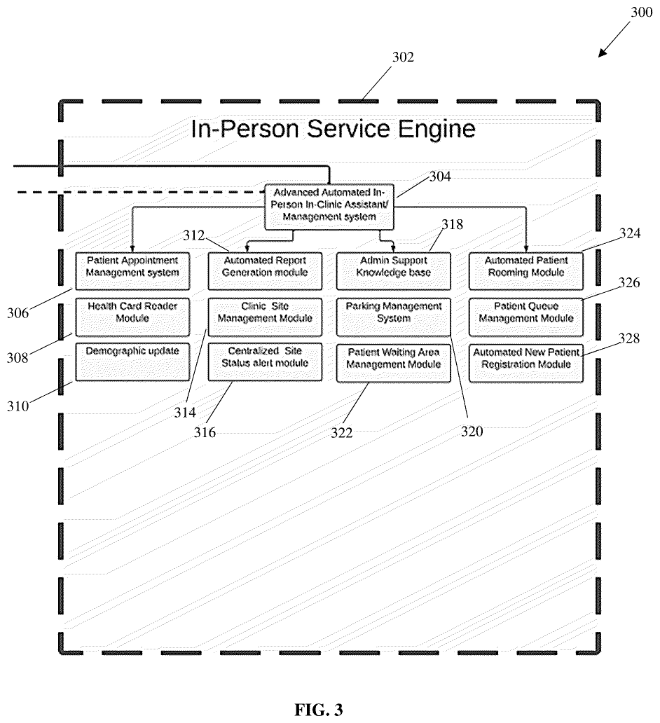

FIG. 3 is a systems diagram illustrating components of the In-Person Service Engine. According to FIG. 3, In-Person Service Engine system 300 is comprised of In-Person Service Engine 302. In-Person Service Engine 302 is identical to In-Person Service Engine 204 in FIG. 2.

According to FIG. 3, In-Person Service Engine system 302 consists of an advanced automated in-person in-clinic assistant or management system 304. Management system 304 is connected to a plurality of components including at least one of the following:
patient appointment management system 306,
health card reader module 308,
demographic update module 310,
automated report generation module 312,
clinic site management module 314,
centralized site status alert module 316,
admin support knowledge base 318,
parking management system 320,
patient waiting area management module 322,
automated patient rooming module 324,
patient queue management module 326, and
automated new patient registration module.

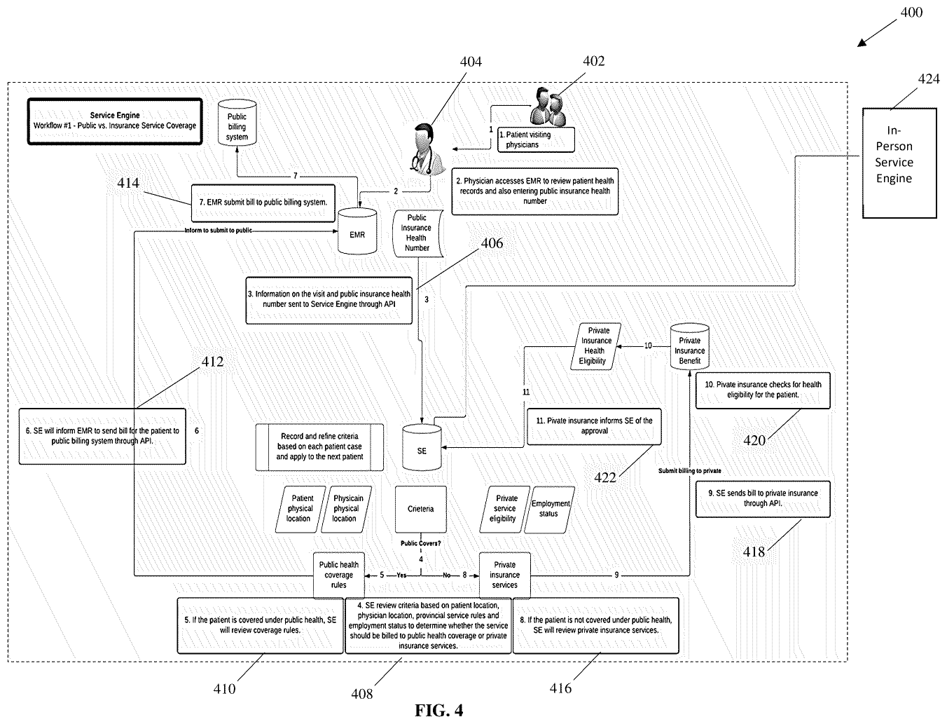

FIG. 4 is a process diagram illustrating a workflow of public vs insurance service coverage using a Service Engine.

According to FIG. 4, the workflow 400 initiates with a patient visiting a physician at step 402. The physician accesses an EMR to review the patient health records and also inputs public insurance health number at step 404. Next, the information on the visit and public insurance health number is sent to the Service Engine (SE) through an application program interface (API) at step 406.

According to FIG. 4 the Service Engine (SE) reviews criteria based on patient location, physician location, provincial service rules and employment status to determine whether the service should be billed to public health coverage or private insurance services at step 408. If the patient is covered under public health insurance, the Service Engine will review coverage rules at step 410. Next, the Service Engine will inform the EMR to send the bill for the patient to the public billing system through the API interface at step 412. Thereafter, the EMR will submit the bill to the public billing system at step 414.

According to FIG. 4, if the patient is not covered under the public health insurance, the Service Engine will review private insurance services at step 416. Next, the Service Engine sends the bill to the private insurance provide via the API interface at step 418. The private insurance checks for health eligibility for the patient at step 420. Finally, the private insurance informs SE of the approval at step 422. Finally, an In-Person Service Engine 424 has a connection to the Service Engine.

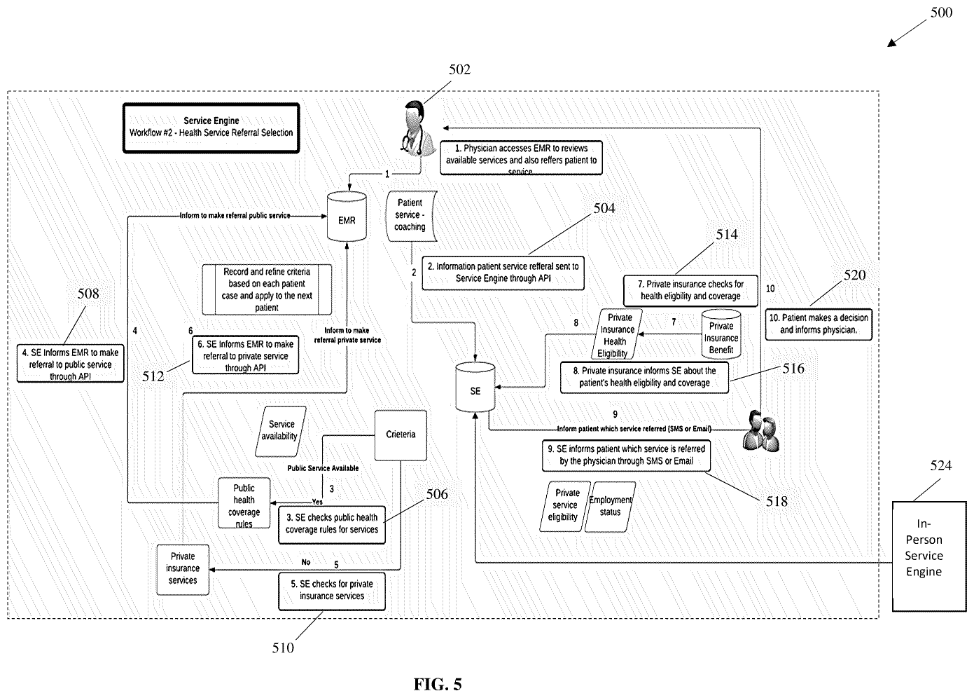

FIG. 5 is a process diagram illustrating a workflow of a health service referral selection. According to FIG. 5, workflow 500 initiates by having a physician access an EMR to review available services and also refers patient to services at step 502. The information patient service referral is sent to the Service Engine (SE) through an API interface at step 504. Next, the Service Engine (SE) checks public health coverage rules for services at step 506. The Service Engine (SE) informs the EMR to make a referral to public service through the API interface at step 508.

According to FIG. 5, the Service Engine checks for private insurance services at step 510. The Service Engine informs the EMR to make a referral to private service through the API interface at step 512. The system records and refines the criteria based on each patient and case and applies to the next patient or future patients.

According to FIG. 5, the private insurance then checks for health eligibility and coverage at step 514. The private insurance informs the Service Engine (SE) about the patient's health eligibility and coverage at step 516. The Service Engine informs the patient which service is referred by the physician through SMS or Email at step 518. Finally, the patient makes a decision and informs physician at step 520. Finally, an In-Person Service Engine 524 has a connection to the Service Engine.

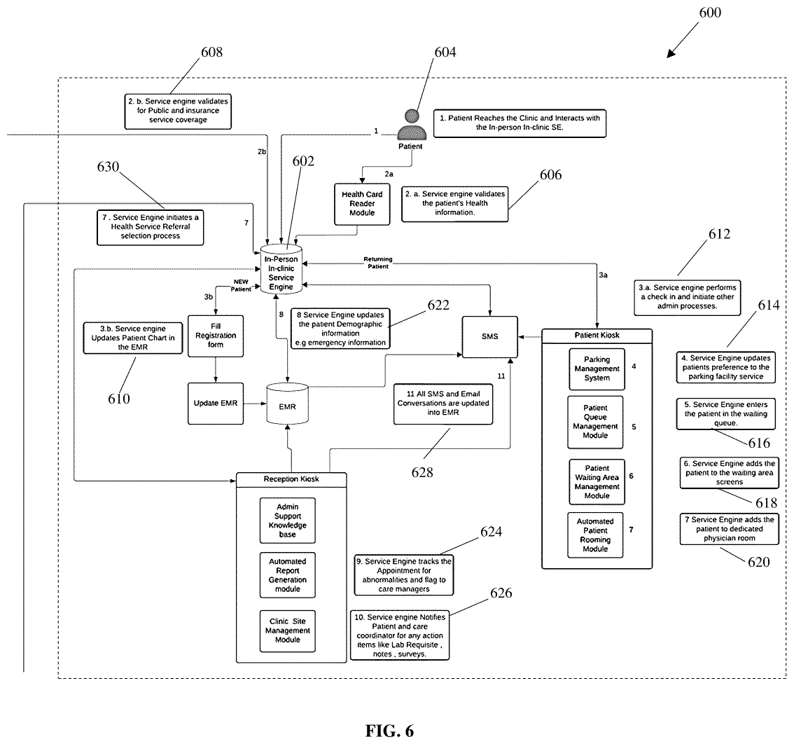

FIG. 6 is a process diagram illustrating a workflow of an In-Person In-clinic Service Engine. According to FIG. 6, workflow 600 initiates when the patient reaches the clinic and interacts at step 604 with the in-person in-clinic service engine 602. The service engine validates the patient's health information at step 606. Furthermore, the service engine also validates for public and insurance service coverage at step 608 which receives input from FIG. 4 and/or the service engine also initiates a Health Service referral selection process at step 630 from FIG. 5.

According to FIG. 6, the service engine updates the patient chart in the EMR at step 610. The service engine also performs check in and initiate other admin processes at step 612. The service engine updates the patient preference to the parking facility service at step 614. The service engine also enters the patient into the waiting queue at step 616. Furthermore, the service engine adds the patient to the waiting area screens at step 618. In further embodiments, the Service Engine (SE) adds patients to the waiting area screen, estimates the average wait time, and provides real-time updates on the waiting screen at step 618. Additionally, notifications are sent to patients via text message to keep them informed.

According to FIG. 6, the service engine also adds the patient to the dedicated physician room at step 620. In further embodiments, the Service Engine (SE) analyzes rooming patterns using daily data from real patient visits to the clinic. This analysis considers various factors. The system is designed to continuously learn and adapt, improving efficiency over time.

According to FIG. 6, the service engine updates the patient demographics information (e.g., emergency information) at step 622. The service engine then tracks the appointment for abnormalities and flags any to the care managers at step 624. In further embodiments, the Service Engine tracks anomalies such as extended wait times, missed patients, and incomplete post-appointment tasks by care coordinators, ensuring a more streamlined and accountable workflow.

According to FIG. 6, the service engine then notifies the patient and care coordinator for relevant actions at step 626. Relevant actions include lab requisitions, blood work, notes and surveys. Finally, all SMS and email conversations are saved and updated into the EMR system at step 628.

According to the disclosure, a service engine system for delivery of virtual care and health care services for patients is disclosed. The service engine system comprises a service engine module, a web portal module, an electronic medical records (EMR) module configured to support EMR systems, and an in-person service engine module further comprising an automated in-person in-clinic management system. The service engine module is configured to integrate the various services and modules and the in-person service engine module to deliver virtual care and real-time health care services for the patient.

According to the disclosure, the service engine system further comprises further modules selected from a list consisting of an appointment booking and reservation module, an email server configured to support one or more email systems, a short messaging service (SMS) server, a payment module configured to support credit card payments, a call center support module configured to support technical support, a fax server configured to support faxing services; and a telephony module configured to support phone services.

According to the disclosure, the in-person service engine module further comprises further modules selected from a list consisting of a patient appointment management system, a health card reader module, a demographic update module, an automated report generation module, a clinic site management module, a centralized site status alert module, an admin support knowledge base module, a parking management system, a patient waiting area management module, an automated patient rooming module, a patient queue management module and an automated new patient registration module.

According to the disclosure, the telephony module of the service engine system supports land-line phones and cellular mobile phones. The service engine system is configured to provide services selected from a list consisting of virtual care appointments, payment collection, pharmacy services, covid testing, utilization analytics, virtual health coaching, insurance group benefits integration, critical diagnostics, care coordination, diagnostics testing.

According to the disclosure, the EMR module of the service engine system is configured to support OscarPro and Ocean EMR systems. The SMS server of the service engine system is configured to support the Swift SMS gateway and the Clinitok SMS gateway.

According to the disclosure, the service engine system is configured to support third party middleware providers for health care procurement. The service engine system is configured to integrate with health service providers including Blue Cross and provincial and state health care providers.

According to the disclosure, a computer-implemented method of managing public and private insurance service coverage using a Service Engine module is disclosed. The method comprises the steps of receiving a patient visiting a physician at a medical clinic, accessing an EMR to review the patient health records, inputting a public insurance health number, sending the information on the visit and public insurance health data to the Service Engine module, reviewing the criteria by the Service Engine module based on patient information, determine whether the service should be billed to public health coverage or private insurance services. If the patient is covered under public health insurance, the Service Engine module is further configured to review coverage rules and inform the EMR to create and send the bill for the patient to the public billing system through the API interface and submit the bill to the public billing system. if the patient is not covered under the public health insurance or has private insurance, the Service Engine module is further configured to review the private insurance services, send the bill to the private insurance provide via the API interface, check for health eligibility for the patient of the private insurance and inform the Service Engine module of the approval of the private insurance.

According to the disclosure, the patient information of the computer-implemented method is selected from a list consisting of patient location, physician location, provincial service rules and employment status. Sending the information on the visit and public insurance health data to the Service Engine module is sent through an application program interface (API).

According to the disclosure, a computer-implemented method of managing a health service referral selection using a Service Engine module is disclosed. The method comprising the steps of accessing an EMR system by a physician to review available services, referring the patient to services sending the information patient service referral to the Service Engine module through an API interface, checking public health coverage rules for services, informing the EMR system to make a referral to public service, checking for private insurance services, informing the EMR to make a referral to private service, checking and informing for health eligibility and coverage, informing patient which service is referred by the physician through SMS or email and having the patient making a decision and informing the physician.

According to the disclosure, the step of sending the information patient service referral to the Service Engine module of the method further comprises the step of informing the EMR system to make a referral to public service or informing the EMR to make a referral to private service is sent through an API interface. The method is configured to record and refine the criteria based on each patient and case and apply to the next patient.

According to the disclosure, a computer-implemented method of managing virtual care and health care service for patients using an in-person in-clinic service engine module is disclosed. The method comprising the steps of receiving a patient at a clinic, interacting with the in-person in-clinic service engine module, validating the patient's health information, validating the patient for public and private insurance service coverage, updating the patient chart in the EMR system, performing check in and initiating other admin processes, updating the patient demographics information, tracking the appointment for abnormalities and flagging these to the care manager and notifying the patient and care coordinator for relevant actions.

According to the disclosure, the step of performing check in and initiating other admin processes of the method is selected from a list consisting of updating the patient preference to the parking facility service, entering the patient in the waiting queue, adding the patient to the waiting area screens and adding the patient to a dedicated physician room.

According to the disclosure, the patient demographics information further comprises emergency information. The relevant actions are selected from a list comprising lab requisitions, blood work, notes and surveys.

Implementations disclosed herein provide systems, methods and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed:

1. A service engine system for delivery of virtual care and health care services for patients comprising:
a service engine module;
a web portal module;
an electronic medical records (EMR) module configured to support EMR systems; and
an in-person service engine module, further comprising:
an automated in-person in-clinic management system;
wherein the service engine module is configured to integrate the various services and modules and the in-person service engine module to deliver virtual care and real-time health care services for the patient.

2. The service engine system of claim 1, further comprising a module selected from a list consisting of:
an appointment booking and reservation module;
an email server configured to support one or more email systems;
a short messaging service (SMS) server;
a payment module configured to support credit card payments;
a call center support module configured to support technical support;
a fax server configured to support faxing services; and
a telephony module configured to support phone services.

3. The service engine system of claim 1 wherein the in-person service engine module further comprises a module selected from a list consisting of:
a patient appointment management system;
a health card reader module;
a demographic update module;
an automated report generation module;
a clinic site management module;
a centralized site status alert module;
an admin support knowledge base module;
a parking management system;
a patient waiting area management module;
an automated patient rooming module;
a patient queue management module; and
an automated new patient registration module.

4. The service engine system of claim 2 wherein the telephony module supports land-line phones and cellular mobile phones.

5. The service engine system of claim 1 wherein the service engine system is configured to provide services selected from a list consisting of virtual care appointments, payment collection, pharmacy services, covid testing, utilization analytics, virtual health coaching, insurance group benefits integration, critical diagnostics, care coordination, diagnostics testing.

6. The service engine system of claim 1 wherein the EMR module is configured to support OscarPro and Ocean EMR systems.

7. The service engine system of claim 1 wherein the SMS server is configured to support the Swift SMS gateway and the Clinitok SMS gateway.

8. The service engine system of claim 1, wherein the service engine system is configured to support third party middleware providers for health care procurement.

9. The service engine system of claim 1, wherein the service engine system is configured to integrate with health service providers including Blue Cross and provincial and state health care providers.

10. A computer-implemented method of managing public and private insurance service coverage using a Service Engine module, the method comprising the steps of:
receiving a patient visiting a physician at a medical clinic;
accessing an EMR to review the patient health records;
inputting a public insurance health number;
sending the information on the visit and public insurance health data to the Service Engine module;
reviewing the criteria by the Service Engine module based on patient information;
determine whether the service should be billed to public health coverage or private insurance services;
if the patient is covered under public health insurance, the Service Engine module is further configured to:
review coverage rules; and
inform the EMR to create and send the bill for the patient to the public billing system through the API interface; and
submit the bill to the public billing system;
if the patient is not covered under the public health insurance or has private insurance, the Service Engine module is further configured to:
review the private insurance services;
send the bill to the private insurance provide via the API interface;
check for health eligibility for the patient of the private insurance; and
inform the Service Engine module of the approval of the private insurance.

11. The computer-implemented method of claim 10 wherein the patient information is selected from a list consisting of patient location, physician location, provincial service rules and employment status.

12. The computer-implemented method of claim 10 wherein sending the information on the visit and public insurance health data to the Service Engine module is sent through an application program interface (API).

13. The computer-implemented method managing a health service referral selection using a Service Engine module, the method comprising the steps of:
accessing an EMR system by a physician to review available services;
referring the patient to services;
sending the information patient service referral to the Service Engine module through an API interface;
checking public health coverage rules for services;
informing the EMR system to make a referral to public service;
checking for private insurance services;
informing the EMR to make a referral to private service;
checking and informing for health eligibility and coverage;
informing patient which service is referred by the physician through SMS or email; and
the patient making a decision and informing the physician.

14. The computer-implemented method of claim 13 wherein
sending the information patient service referral to the Service Engine module;
informing the EMR system to make a referral to public service, or
informing the EMR to make a referral to private service is sent through an API interface.

15. The computer-implemented method of claim 13 wherein the method is configured to record and refine the criteria based on each patient and case and apply to the next patient.

16. A computer-implemented method of managing virtual care and health care service for patients using an in-person in-clinic service engine module, the method comprising the steps of:
receiving a patient at a clinic;
interacting with the in-person in-clinic service engine module;
validating the patient's health information;
validating the patient for public and private insurance service coverage;
updating the patient chart in the EMR system;
performing check in and initiating other admin processes;
updating the patient demographics information;
tracking the appointment for abnormalities and flagging these to the care manager; and
notifying the patient and care coordinator for relevant actions.

17. The computer-implemented method of claim 16 wherein performing check in and initiating other admin processes is selected from a list consisting of:
updating the patient preference to the parking facility service;
entering the patient in the waiting queue;
adding the patient to the waiting area screens; and
adding the patient to a dedicated physician room.

18. The computer-implemented method of claim 16 wherein patient demographics information further comprises emergency information.

19. The computer-implemented method of claim 16 wherein relevant actions is selected from a list comprising lab requisitions, blood work, notes and surveys.
